(19) United States
(12) Patent Application Publication
(10) Pub. No.: US 2018/0149627 A1
(43) Pub. Date: May 31, 2018

(54) LOW PRESSURE ANION EXCHANGE CHROMATOGRAPHY-TURBIDIMETRIC METHOD FOR SIMULTANEOUS ONLINE ANALYSIS OF TRACE SULFIDE AND CHLORIDE IN WATER SAMPLES

(71) Applicant: SICHUAN UNIVERSITY, Chengdu (CN)

(72) Inventors: Xinshen ZHANG, Chengdu (CN); Zhengxi ZHAO, Chengdu (CN); Yuexin GAO, Chengdu (CN); Shan MO, Chengdu (CN)

(73) Assignee: SICHUAN UNIVERSITY, Chengdu (CN)

(21) Appl. No.: 15/823,626

(22) Filed: Nov. 28, 2017

(30) Foreign Application Priority Data
Nov. 29, 2016 (CN) 201611072725.7

(51) Int. Cl. G01N 30/74; G01N 30/52; G01N 30/32; G01N 33/18; G01N 21/78

(52) U.S. Cl. CPC G01N 30/74; G01N 30/52; G01N 2030/525; G01N 33/182; G01N 21/78; G01N 30/32

(57) ABSTRACT

The present invention provides a low pressure anion exchange chromatography—turbidimetric method for simultaneous online analysis of trace S²⁻ and Cl⁻ in water samples using an apparatus comprising a low pressure pump, a sample valve, a sample loop, a low pressure anion chromatographic column, a reactor, an optical flow cell, an optical detector, a computer system, a mixer, a sample flow path, a propelling solution flow path, and a color developer solution flow path, the method comprising: (a) mapping a baseline; (b) mapping spectrogram of S²⁻ and Cl⁻ in test samples; (c) mapping standard working curves; and (d) calculating the concentrations of S²⁻ and Cl⁻ in the test samples based on the peak heights of S²⁻ and Cl⁻ in the spectrogram and the regression equations of standard working curves. In this method, a chromatography method is combined with a turbidimetric method for the first time to realize simultaneous online analysis of trace S²⁻ and Cl⁻ in water samples, and the method is endowed with the advantages of fast analysis speed, high analysis efficiency and low analysis costs.

### FIELD OF THE INVENTION

The present invention relates to assay and analysis of chlorides and sulfides, and in particular to a method for automatic simultaneous online analysis of trace S²⁻ and Cl⁻ in water samples.

### BACKGROUND OF THE INVENTION

Chlorides and sulfides are two major sources of contamination in tanning industry. Both sodium chloride used in the procedures of salting, soaking, pickling, chrome tanning and the like and ammonium chloride used in the procedure of deliming may result in a high content of chlorides in tannery wastewater, and the procedures of unhairing and liming may lead to the production of a large amount of sulfide-containing wastewater. Highly reactive chlorine ions (Cl⁻) and sulfur ions (S²⁻) may cause damage to the oxide protective film on metal surface, which not only subjects ferrous metal and nonferrous metal to pitting corrosion, but also accelerates the rusting of reinforcing steel bars in concrete and thus affects their durability. Chlorine ions in a high concentration may run off along with water after complexing with calcium ions in soil, and thus result in soil hardening. Moreover, chlorine ions and sulfur ions in water may also affect the taste and smell of water, and when their concentrations are excessively high, plants, fishes and aquatic organisms will be jeopardized. Also, hydrogen sulfide is a neurotoxic gas having a rotten egg smell.

Sulfides and chlorides are used in large quantities in the process of tanning, and the contents of both chlorine ions and sulfur ions in tannery wastewater are far beyond the concentrations in state-specified discharge standards. In order to control the contamination caused to the environment by chlorine ions and sulfur ions in tannery wastewater, the concentrations of chlorine ions and sulfur ions in tannery wastewater need to be assayed and analyzed frequently in production practice. Currently, chlorine ions are generally measured using an electrode method, a titrimetric method or a turbidimetric method. While both chlorine ions and sulfur ions can form precipitates with silver ions, sulfur ions cannot be analyzed by a turbidimetric method since they react quickly with silver ions and form black precipitates of large silver sulfide particles. Therefore, sulfur ions are generally analyzed using an amperomatric method or an ion chromatographic method. In the above methods, the assay of chlorine ions and sulfur ions requires separate sampling and separate analysis, leading to drawbacks such as slow analysis speed, poor analysis efficiency and a large amount of labor for operators.

CN 101551367 A (corresponding to United States Patent Publication No. 2010/0267159) discloses a low pressure ion-exclusion chromatography-catalytic kinetic spectrophotometric method for simultaneous analysis of chlorides and sulfides. Although the method realizes simultaneous online analysis of Cl⁻ and S²⁻, it has the following drawbacks: as the catalytic kinetic spectrophotometric method is based on ion-exclusion chromatography and conducted in sulfuric acid of a high concentration using sodium nitrate in a concentration of 10 mmol/L as an eluent, two sets of color developer solutions are required, and the two sets of color developer solutions need to react with Cl⁻ and S²⁻ at 75-85° C., thus the corresponding apparatus is required to be equipped with two color developer solution flow paths and also with heating and temperature-controlling units, which is disadvantageous in terms of reduction in apparatus costs and simplification of analysis flow paths.

### SUMMARY OF THE INVENTION

An object of the present invention is to overcome the disadvantages of the prior art, and provide a low pressure anion exchange chromatography—turbidimetric method for simultaneous online analysis of trace S²⁻ and Cl⁻ in water samples so as to reduce analysis costs of S²⁻ and Cl⁻ and simplify analysis operations.

The present invention provides a low pressure anion exchange chromatography—turbidimetric method for simultaneous online analysis of trace S²⁻ and Cl⁻ in water samples using an apparatus comprising a low pressure pump, a sample valve, a sample loop, a low pressure anion chromatographic column, a reactor, an optical flow cell, an optical detector, a computer system, a mixer, a sample flow path, a propelling solution flow path, and a color developer solution flow path, the method comprising:

(a) setting the apparatus in sample injection state, in which a blank sample is driven by the low pressure pump to enter the sample loop through the sample flow path and the sample valve; and then setting the apparatus in analytical state, in which a color developer solution is driven by the low pressure pump to enter the mixer through the color developer solution flow path, a propelling solution is driven by the low pressure pump to enter the sample loop through the propelling solution flow path and the sample valve, the propelling solution brings the blank sample in the sample loop to enter the mixer through the low pressure anion chromatographic column, they are mixed with the color developer solution in the mixer, then the mixture enters the optical flow cell through the reactor, and signals produced via the optical detector are transferred to the computer system for processing to obtain a baseline;

(b) setting the apparatus in sample injection state, in which a test sample is driven by the low pressure pump to enter the sample loop through the sample flow path and the sample valve; and then setting the apparatus in analytical state, in which the color developer solution is driven by the low pressure pump to enter the mixer through the color developer solution flow path, the propelling solution is driven by the low pressure pump to enter the sample loop through the propelling solution flow path and the sample valve, the propelling solution brings the test sample in the sample loop to enter the low pressure anion chromatographic column, S²⁻ and Cl⁻ in the test sample, after being separated in the low pressure anion chromatographic column, enters the mixer successively, where they are mixed with the color developer solution respectively to form a first mixed solution and a second mixed solution, which enter the reactor successively and form a first reaction solution and a second reaction solution upon color development reactions, the first reaction solution and the second reaction solution enter the optical flow cell successively, and signals produced via the optical detector are transferred to the computer system for processing to obtain spectrogram of S²⁻ and Cl⁻ in the test sample;

(c) repeating steps (a) and (b) except for replacing the test sample with a series of standard samples, in which the concentrations of S²⁻ and Cl⁻ are known, to obtain spectrogram of S²⁻ and Cl⁻ in the standard samples, and mapping standard working curves with the concentrations of S²⁻ and Cl⁻ in the standard samples being abscissa and the peak heights of S²⁻ and Cl⁻ in the spectrogram of S²⁻ and Cl⁻ in the standard samples being ordinate; and

(d) calculating the concentrations of S²⁻ and Cl⁻ in the test sample by substituting the peak heights of S²⁻ and Cl⁻ in the spectrogram of S²⁻ and Cl⁻ in the test sample into the regression equations of the standard working curves obtained in step (c), respectively; wherein the test sample and the standard samples comprise NaOH in a concentration of from 10⁻⁵ mmol/L to 10⁻³ mmol/L; the blank sample is an aqueous solution of NaOH in a concentration of from 10⁻⁵ mmol/L to 10⁻³ mmol/L; the propelling solution is a mixed solution of nitric acid, sodium nitrate and deionized water; and the color developer solution is a mixed solution of silver nitrate, polyvinylpyrrolidone K-30, gelatin, nitric acid and deionized water.

Preferably, the column filler of the low pressure anion chromatographic column is strongly basic quaternary ammonium anion exchange resin. The particle size of the column filler is 30-35 m, and the exchange capacity of the column filler is 3-4 mmol/g. The strongly basic quaternary ammonium anion exchange resin is prepared according to the method disclosed in Chapter 2 of “Ion Exchange and Absorption Resin [M].” at pages 43-50 (He Binglin and Huang Wenqiang, published by Shanghai Science and Technology Education Press in 1995).

Preferably, the propelling solution comprises nitric acid in a concentration of 1-10 mmol/L (e.g., 1, 2, 3, 4, 5, 6, 7, 8, 9 or 10 mmol/L), and sodium nitrate in a concentration of 1.0-10.0 g/L (e.g., 1.0, 2.0, 3.0, 4.0, 5.0, 6.0, 7.0, 8.0, 9.0 or 10.0 g/L).

Preferably, the color developer solution comprises nitric acid in a concentration of 0.10-0.50 mol/L (e.g., 0.10, 0.20, 0.30, 0.40 or 0.50 mol/L), silver nitrate in a concentration of 0.10-0.50 g/L (e.g., 0.10, 0.20, 0.30, 0.40 or 0.50 g/L), polyvinylpyrrolidone K-30 in a concentration of 0.10-0.50 g/L (e.g., 0.10, 0.20, 0.30, 0.40 or 0.50 g/L), and gelatin in a concentration of 0.50-1.0 g/L (e.g., 0.50, 0.60, 0.70, 0.80, 0.90 or 1.0 g/L).

Preferably, the detection wavelength of the optical detector is 420 nm.

Preferably, the optical path of the optical flow cell is 20-30 mm.

Preferably, the test sample is filtrated by a microporous membrane and subjected to decolorization by macroporous adsorption resin before entering the low pressure pump.

The present invention provides a new method for simultaneous online analysis of S²⁻ and Cl⁻ in water samples, which has the following beneficial effects:

1. A chromatographic method is combined with a turbidimetric method for the first time, and as the strongly acidic anion Cl⁻ is present in a form completely different from that of the weakly acidic anion S²⁻ under acidic conditions, anion resin is employed to achieve rapid separation of the two anions using an acidic sodium nitrate solution as an eluent (the propelling solution). The color developer solution in the prior turbidimetric method is modified, thereby realizing simultaneous online analysis of S²⁻ and Cl⁻. Compared with the method of separate sampling and separate assay that is commonly employed at present, the present method has the advantages of fast analysis speed, high analysis efficiency and simplified operations. Compared with the prior method for simultaneous assay of S²⁻ and Cl⁻, it is not necessary for the apparatus to be equipped with heating and temperature-controlling units, and the analysis flow paths are simpler. The apparatus is quite low costing, having the advantages of lower analysis costs and simpler operations.

2. Assay of S²⁻ and Cl⁻ is carried out using a turbidimetric method after they are separated with a low pressure anion chromatographic column. As a dispersant (polyvinylpyrrolidone K-30) and a suspending agent (gelatin) are added into the color developer solution of the prior turbidimetric method, and the concentrations of the various components in the color developer solution are properly arranged, precipitation of silver chloride and silver sulfide can be effectively avoided, such that chlorine ions and sulfur ions react with silver ions to form a colloidal solution with excellent stability and dispersibility. As such, the interference of Cl⁻ with the assay of S²⁻ by a turbidimetric method is reduced, and vice versa. The reproducibility of analysis and assay is significantly improved, and thus the accuracy of analysis and assay of S²⁻ and Cl⁻ is improved (see Examples 1 and 2).

3. The present method has good precision. A mixed standard sample of 5 mg/L of sulfides and 50 mg/L of chlorides are measured repeatedly for 10 times, and the relative standard deviation of the peak heights of chlorine ions in the spectrogram is 2.30%, while the relative standard deviation of the peak heights of sulfur ions in the spectrogram is 0.96%.

4. The quantitative detection limits for S²⁻ and Cl⁻ are 3.47 mg/L and 0.04 mg/L, respectively. The standard working curves obtained in the concentration range of sulfur ions of 0.2-15 mg/L and in the concentration range of chlorine ions of 5-150 mg/L have good linearity, and are particularly suitable for the assay of S²⁻ and Cl⁻ contents in tannery wastewater.

5. The recovery rate is in the range of 85-115%, which is close to the value measured using the national standard method and the recovery rate of standard addition.

6. The present method is endowed with the advantages of simple operations, fast analysis speed and low costs, which are advantageous for its promotion and application.

### BRIEF DESCRIPTION OF THE DRAWINGS

FIG. 1 is a schematic illustration of the process flow diagram of the present method in the sample injection state and of the structure of the corresponding apparatus.

FIG. 2 is a schematic illustration of the process flow diagram of the present method in the analytical state and of the structure of the corresponding apparatus.

The symbols in the drawings are as follows: 1—low pressure pump, 2—sample valve, 3—sample loop, 4—low pressure anion chromatographic column, 5—reactor, 6—optical flow cell, 7—optical detector, 8—computer system, 9—mixer, C—propelling solution, R—color developer solution, S₀—blank sample, S₁—test sample, S₂—standard sample, W—waste fluid.

FIG. 3 is the spectrogram of S²⁻ and Cl⁻ from the experiment in which a color developer solution free of dispersant and suspending agent is employed in Example 1.

FIG. 4 is the spectrogram of S²⁻ and Cl⁻ from the experiment in which a color developer solution containing dispersant and suspending agent is employed in Example 1.

FIG. 5 is the spectrogram showing the precision in the assay of standard samples of S²⁻ and Cl⁻ in Example 2.

FIG. 6 is a diagram illustrating the standard working curves of standard samples of S²⁻ and Cl⁻ mapped in Example 3.

### DETAILED DESCRIPTION OF THE INVENTION

The present invention will be further illustrated below by way of examples and with reference to the accompanying drawings. These examples are meant to be only illustrations of the present invention and do not limit it.

### Example 1: Effect of the Composition of the Color Developer Solution on the Assay of S²⁻ and Cl⁻ by the Turbidimetric Method

The effect of the composition of the color developer solution on the assay of S²⁻ and Cl⁻ by the turbidimetric method was studied.

1. Preparation of Standard Samples of Cl⁻ and S²⁻

Standard stock solution of chlorine ions (1000 mg/L): 0.1667 g of sodium chloride was weighed and transferred into a 100-mL volumetric flask, and the volume was brought up to the graduation mark with deionized water. The solution was evenly mixed by shaking, and for use, an appropriate volume was taken and diluted stepwise with deionized water to desired concentrations.

Standard stock solution of sulfur ions (1000 mg/L): A certain amount of crystalline sodium sulfide nonahydrate was taken and placed into a 50-mL small beaker or a Buchner funnel, rinsed with deionized water repeatedly to remove impurities on the surface. Then the moisture was absorbed immediately by clean filter paper, and 0.7506 g of the resulting crystal was rapidly weighed and dissolved in a small amount of deionized water. The solution was transferred into a 100-mL brown volumetric flask, and the volume was brought up to the graduation mark with deionized water. The solution was evenly mixed by shaking, and was placed in a refrigerator at 4° C. and protected from light. For use, an appropriate volume was taken and diluted stepwise with deionized water to desired concentrations.

Standard sample of sulfur ions in a concentration of 5 mg/L (pH 9-11): 0.5 mL of the standard stock solution of sulfur ions was transferred into a 100-mL volumetric flask. The pH value was adjusted to 9-11 using a standard sodium hydroxide solution, and the volume was brought up to the graduation mark with deionized water.

Standard sample of chlorine ions in a concentration of 50 mg/L (pH 9-11): 5 mL of the standard stock solution of chlorine ions was transferred into a 100-mL volumetric flask. The pH value was adjusted to 9-11 using a standard sodium hydroxide solution, and the volume was brought up to the graduation mark with deionized water.

### 2. Preparation of a Blank Sample

The pH value of deionized water was adjusted to 9-11 using a standard sodium hydroxide solution to obtain the blank sample.

### 3. Preparation of a Propelling Solution C

1 g of sodium nitrate was dissolved in deionized water. 1 mL of a nitric acid solution (1 mol/L) was added, and then the volume was brought up to 1 L in a volumetric flask to obtain the propelling solution C.

### 4. Preparation of a Color Developer Solution R Free of Dispersant and Suspending Agent

0.4 g of silver nitrate was weighed, and 400 mL of a nitric acid solution (1 mol/L) was added. The volume was brought up to 1 L with deionized water in a volumetric flask to obtain the color developer solution R free of dispersant and suspending agent.

### 5. Preparation of a Color Developer Solution R Containing Dispersant and Suspending Agent

0.1 g of dispersant (polyvinylpyrrolidone K-30), 1 g of suspending agent (gelatin) and 0.4 g of silver nitrate were weighed, and 400 mL of a nitric acid solution (1 mol/L) was added. The volume was brought up to 1 L with deionized water in a volumetric flask to obtain the color developer solution R containing dispersant and suspending agent.

### 6. Experiment Conducted Using the Color Developer Solution R Free of Dispersant and Suspending Agent

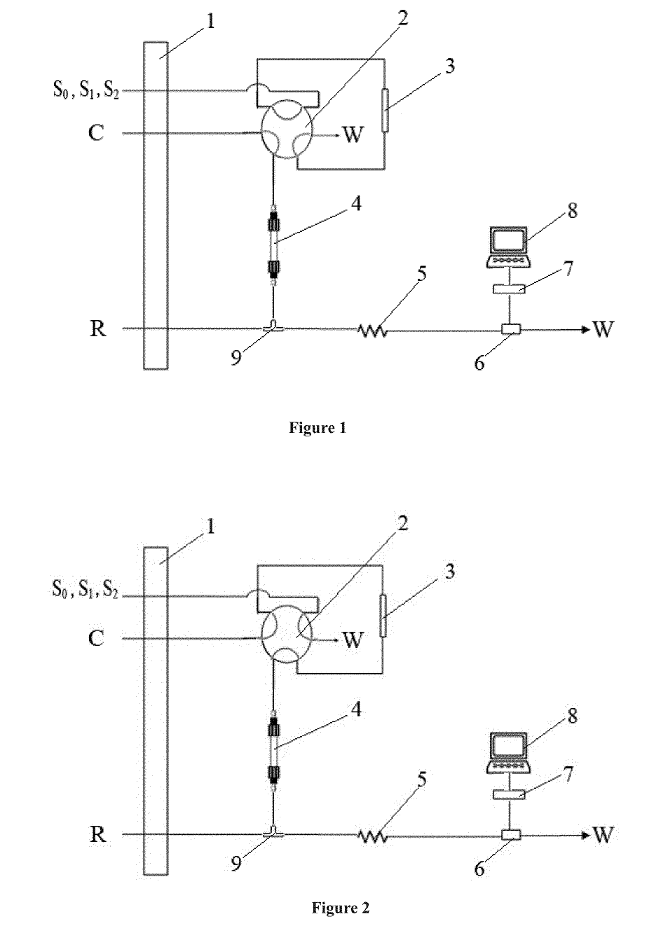

The experiment was carried out using an apparatus as shown in FIGS. 1 and 2 except for the absence of the low pressure anion chromatographic column 4. In this apparatus, the low pressure pump 1 was a four-channel constant-flow peristaltic pump, with a pump capacity of 0.4-1.0 mL/min and a working pressure of 2×10⁵-3×10⁵ Pa; the sample valve 2 was a six-channel automatic sample valve, wherein the sample injection state of the six-channel automatic sample valve was adjusted using a time relay; the sample loop 3 was a polytetrafluoroethylene tube, the specific volume of which may be learned from calculation, wherein the inner diameter and length of the tube may be modified to adjust its volume; the reactor 5 was of a coil structure, and was made by winding polytetrafluoroethylene tubes having an inner diameter of 0.5 mm and a length of 3.0 m; the optical path of the optical flow cell 6 was 28 mm, and the detection wavelength of the optical detector 7 was adjusted to be 420 nm; the computer system 8 was a personal computer installed with an HW-2000 chromatographic workstation (Shanghai Qianpu Software Co., Ltd.); the optical flow cell of this apparatus was connected with a waste fluid container, and the sample was discharged into the waste fluid container after flowing through the optical flow cell.

(a) The apparatus was set in sample injection state. Driven by the low pressure pump 1, the blank sample S₀ entered the sample loop 3 through the sample flow path and the sample valve 2 and filled the sample loop. The redundant sample was discharged into the waste fluid container in the form of waste fluid W. The color developer solution R free of dispersant and suspending agent entered the mixer 9 through the color developer solution flow path. The propelling solution C entered the mixer 9 through the propelling solution flow path and the sample valve 2 to be mixed with the color developer solution R free of dispersant and suspending agent and then entered the optical flow cell 6 through the reactor 5. Then the apparatus was switched to analytical state. Driven by the low pressure pump 1, the blank sample S₀ was discharged into the waste fluid container in the form of waste fluid W after passing through the sample flow path and the sample valve 2. The color developer solution R free of dispersant and suspending agent entered the mixer 9 through the color developer solution flow path. The propelling solution C entered the sample loop 3 through the propelling solution flow path and the sample valve 2. The propelling solution brought the blank sample in the sample loop 3 to enter the mixer 9 where they were mixed with the color developer solution R free of dispersant and suspending agent. Then the mixture entered the optical flow cell 6 through the reactor 5. Signals produced via the optical detector 7 were transferred to the computer system 8 for processing to obtain a baseline.

(b) The apparatus was set in sample injection state. Driven by the low pressure pump 1, the standard sample of sulfur ions entered the sample loop 3 through the sample flow path and the sample valve 2 and filled the sample loop. The redundant standard sample of sulfur ions was discharged into the waste fluid container in the form of waste fluid W. The color developer solution R free of dispersant and suspending agent entered the mixer 9 through the color developer solution flow path. The propelling solution C entered the mixer 9 through the propelling solution flow path and the sample valve 2 to be mixed with the color developer solution R free of dispersant and suspending agent and then entered the optical flow cell 6 through the reactor 5. Then the apparatus was switched to analytical state. Driven by the low pressure pump 1, the standard sample of sulfur ions was discharged into the waste fluid container in the form of waste fluid W after passing through the sample flow path and the sample valve 2. The color developer solution R free of dispersant and suspending agent entered the mixer 9 through the color developer solution flow path. The propelling solution C entered the sample loop 3 through the propelling solution flow path and the sample valve 2. The propelling solution brought the standard sample of sulfur ions in the sample loop 3 to enter the mixer 9 where they were mixed with the color developer solution R free of dispersant and suspending agent. Then the mixture entered the optical flow cell 6. Signals produced via the optical detector 7 were transferred to the computer system 8 for processing to obtain a spectrogram of the standard sample of sulfur ions.

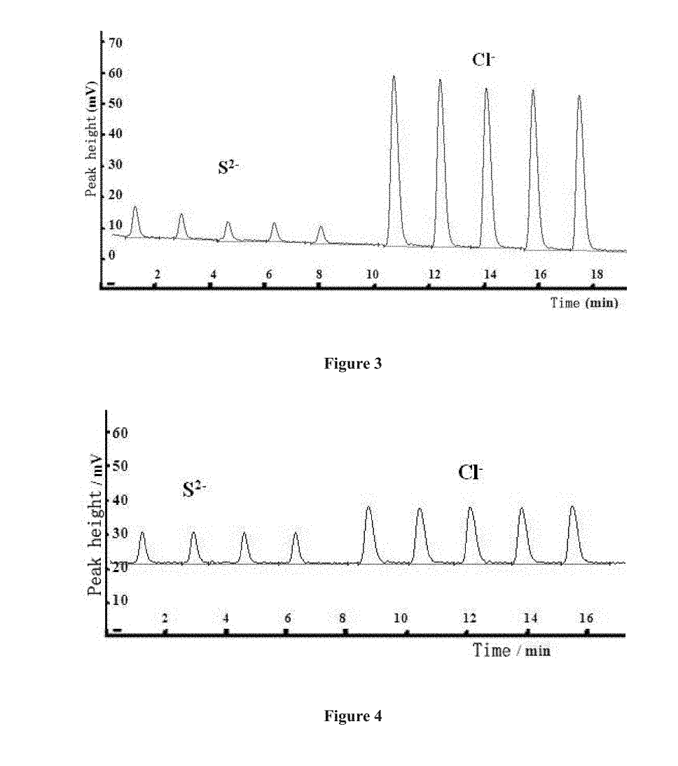

The above steps (a) and (b) were repeated for 5 times in total. Then steps (a) and (b) were repeated for 5 times with the standard sample of sulfur ions being replaced with the standard sample of chlorine ions. The spectrogram as shown in FIG. 3 was obtained.

### 7. Experiment Conducted Using the Color Developer Solution R Containing Dispersant and Suspending Agent

The experiment was carried out with the color developer solution R free of dispersant and suspending agent in step 6 being replaced with the color developer solution R containing dispersant and suspending agent. The spectrogram as shown in FIG. 4 was obtained.

As can be seen from the comparison between FIG. 3 and FIG. 4, the peak heights of S²⁻ and Cl⁻ in the spectrogram decreased gradually and the reproducibility was quite poor when the color developer solution is free of dispersant (polyvinylpyrrolidone K-30) and suspending agent (gelatin), while the peak heights of S²⁻ and Cl⁻ in the spectrogram were stable, the reproducibility was significantly improved, and the analysis precision was remarkably increased when the color developer solution contains dispersant (polyvinylpyrrolidone K-30) and suspending agent (gelatin). This is because the colloidal solution of silver chloride and silver sulfide formed when the dispersant polyvinylpyrrolidone K-30 and the suspending agent gelatin were added into the color developer solution had very good stability and dispersibility.

### Example 2: Precision of the Present Method

Standard samples were tested in order to study the precision of the present method, and the steps are as follows:

1. Preparation of Standard Samples Containing Cl⁻ and S²⁻

A standard stock solution of chlorine ions (1000 mg/L) and a standard stock solution of sulfur ions (1000 mg/L) were prepared according to step 1 of Example 1.

Mixed standard sample of sulfur ions in a concentration of 5 mg/L and chlorine ions in a concentration of 50 mg/L (pH 9-11): 5 mL of the standard stock solution of chlorine ions and 0.5 mL of the standard stock solution of sulfur ions were transferred into a 100-mL volumetric flask. The pH value was adjusted to 9-11 using a standard sodium hydroxide solution, and the volume was brought up to the graduation mark with deionized water.

### 2. Preparation of a Blank Sample

The blank sample was prepared according to step 2 of Example 1.

### 3. Preparation of a Propelling Solution C

10 g of sodium nitrate was dissolved in deionized water. 10 mL of a nitric acid solution (1 mol/L) was added, and then the volume was brought up to 1 L in a volumetric flask to obtain the propelling solution C.

### 4. Preparation of a Color Developer Solution R

0.1 g of polyvinylpyrrolidone K-30, 1 g of gelatin and 0.4 g of silver nitrate were weighed, and 400 mL of a nitric acid solution (1 mol/L) was added. The volume was brought up to 1 L with deionized water in a volumetric flask to obtain the color developer solution R.

### 5. Mapping of Spectrogram of Standard Samples

The experiment was carried out using the apparatus as shown in FIGS. 1 and 2. In this apparatus, the low pressure pump 1 was a four-channel constant-flow peristaltic pump, with a pump capacity of 0.4-1.0 mL/min and a working pressure of 2×10⁵-3×10⁵ Pa; the sample valve 2 was a six-channel automatic sample valve, wherein the sample injection state of the six-channel automatic sample valve was adjusted using a time relay; the sample loop 3 was a polytetrafluoroethylene tube, the specific volume of which may be learned from calculation, wherein the inner diameter and length of the tube may be modified to adjust its volume; the column filler of the low pressure anion chromatographic column 4 was strongly basic quaternary ammonium anion exchange resin, wherein the particle size of the column filler was 30-35 μm, and the exchange capacity of the column filler was 3-4 mmol/g; the reactor 5 was of a coil structure, and was made by winding polytetrafluoroethylene tubes having an inner diameter of 0.5 mm and a length of 3.0 m: the optical path of the optical flow cell 6 was 28 mm, and the detection wavelength of the optical detector 7 was adjusted to be 420 nm; the computer system 8 was a personal computer installed with an HW-2000 chromatographic workstation (Shanghai Qianpu Software Co., Ltd.); the optical flow cell of this apparatus was connected with a waste fluid container, and the sample was discharged into the waste fluid container after flowing through the optical flow cell.

(a) The apparatus was set in sample injection state. Driven by the low pressure pump 1, the blank sample S₀ entered the sample loop 3 through the sample flow path and the sample valve 2 and filled the sample loop. The redundant sample was discharged into the waste fluid container in the form of waste fluid W. The color developer solution R entered the mixer 9 through the color developer solution flow path. The propelling solution C entered the mixer 9 through the propelling solution flow path, the sample valve 2 and the low pressure anion chromatographic column 4 to be mixed with the color developer solution R and then entered the optical flow cell 6 through the reactor 5. Then the apparatus was switched to analytical state. Driven by the low pressure pump 1, the blank sample S₀ was discharged into the waste fluid container in the form of waste fluid W after passing through the sample flow path and the sample valve 2. The color developer solution R entered the mixer 9 through the color developer solution flow path. The propelling solution C entered the sample loop 3 through the propelling solution flow path and the sample valve 2. The propelling solution brought the blank sample in the sample loop 3 to enter the mixer 9 through the low pressure anion chromatographic column 4. They were mixed with the color developer solution R in the mixer 9. Then the mixture entered the optical flow cell 6 through the reactor 5. Signals produced via the optical detector 7 were transferred to the computer system 8 for processing to obtain a baseline.

(b) The apparatus was set in sample injection state. Driven by the low pressure pump 1, the standard sample S₂ entered the sample loop 3 through the sample flow path and the sample valve 2 and filled the sample loop. The redundant standard sample S₂ was discharged into the waste fluid container in the form of waste fluid W. The color developer solution R entered the mixer 9 through the color developer solution flow path. The propelling solution C entered the mixer 9 through the propelling solution flow path, the sample valve 2 and the low pressure anion chromatographic column 4 to be mixed with the color developer solution R and then entered the optical flow cell 6 through the reactor 5. Then the apparatus was switched to analytical state. Driven by the low pressure pump 1, the standard sample S₂ was discharged into the waste fluid container in the form of waste fluid W after passing through the sample flow path and the sample valve 2. The color developer solution R entered the mixer 9 through the color developer solution flow path. The propelling solution C entered the sample loop 3 through the propelling solution flow path and the sample valve 2. The propelling solution brought the standard sample S₂ in the sample loop 3 to enter the low pressure anion chromatographic column 4. S²⁻ and Cl⁻ in the standard sample S₂, after being separated in the low pressure anion chromatographic column 4, is brought by the propelling solution to enter the mixer 9 successively, where they were mixed with the color developer solution R respectively to form a first mixed solution and a second mixed solution, which entered the reactor 5 successively and formed a first reaction solution and a second reaction solution upon color development reactions. The first reaction solution and the second reaction solution entered the optical flow cell 6 successively, and signals produced via the optical detector 7 were transferred to the computer system 8 for processing to obtain spectrogram of S²⁻ and Cl⁻ in the standard sample S₂.

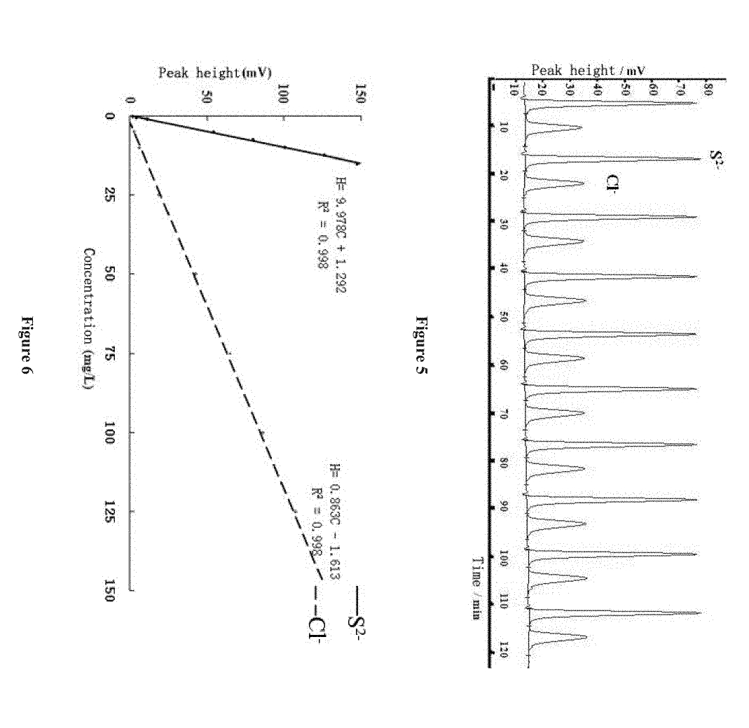

The above steps (a) and (b) were repeated for 10 times in total. The spectrogram as shown in FIG. 5 was obtained. When a mixed standard sample of 5 mg/L of sulfides and 50 mg/L of chlorides was measured using the present method, the relative standard deviation of the peak heights of chlorine ions in the spectrogram was 2.30%, and the relative standard deviation of the peak heights of the sulfur ions in the spectrogram was 0.96%. This indicates that the method of the present invention has good precision.

### Example 3: Mapping of a Standard Working Curve

Standard working curves were mapped, and the steps were as follows:

### 1. Preparation of a Standard Sample and a Blank Sample

(1) A standard stock solution of chlorine ions (1000 mg/L) was prepared according to step 1 of Example 1.

(2) A standard stock solution of sulfur ions (1000 mg/L) was prepared according to step 1 of Example 1.

(3) Preparation of standard sample series of chlorine ions: the standard stock solution of chlorine ions prepared in step (1) was diluted with deionized water, and the pH value was adjusted to 9-11 using sodium hydroxide, to prepare standard sample No. 1 to No. 9, in which the concentrations of chlorine ions were 0 mg/L, 5 mg/L, 10 mg/L, 25 mg/L, 50 mg/L, 75 mg/L, 100 mg/L, 125 mg/L and 150 mg/L, respectively, and the pH values of standard sample No. 1 to No. 9 were all 9-11.

(4) Preparation of standard sample series of sulfur ions: the standard stock solution of sulfur ions prepared in step (2) was diluted with deionized water, and the pH value was adjusted to 9-11 using sodium hydroxide, to prepare standard sample No. 10 to No. 18, in which the concentrations of sulfur ions were 0 mg/L, 0.2 mg/L, 0.5 mg/L, 1 mg/L, 5 mg/L, 7.5 mg/L, 10 mg/L, 12.5 mg/L and 15 mg/L, respectively, and the pH values of standard sample No. 10 to No. 18 were all 9-11.

(5) A blank sample was prepared according to step 2 of Example 1.

### 2. Preparation of a Propelling Solution C

The propelling solution C was prepared according to step 3 of Example 2.

### 3. Preparation of a Color Developer Solution R

The color developer solution R was prepared according to step 4 of Example 2.

### 4. Mapping of Spectrogram of Standard Samples

Steps (a) and (b) according to Step 5 of Example 2 were performed using each of standard sample No. 1 to No. 18 instead of the standard sample S₂ in Example 2 to obtain spectrogram of S²⁻ and Cl⁻ of each standard sample. The standard working curve of chlorine ions was mapped with the concentrations of chlorine ions (mg/L) in the standard samples being abscissa and the peak heights (mV) of chlorine ion in the spectrogram being ordinate, and the standard working curve of sulfur ions was mapped with the concentrations of sulfur ions (mg/L) in the standard samples being abscissa and the peak heights (mV) of sulfur ions in the spectrogram being ordinates. When the concentration of chlorine ions was within the linear range of 5-150 mg/L and the concentration of sulfur ions was within the linear range of 0.2-15 mg/L, the standard working curves were as shown in FIG. 6. The regression equation obtained from the standard working curve of chlorine ions was H=0.863C-1.613, R²=0.998; and the regression equation obtained from the standard working curve of sulfur ions was H=9.978C+1.292, R²=0.998 (wherein H is the peak height (mV); C is the concentration (mg/L) of substances to be assayed in the standard sample). Based on the baseline noise of the apparatus, a three-fold signal-noise ratio was taken as the quantitative detection limit, and the quantitative detection limits of the present method for Cl⁻ and S²⁻ were calculated to be 3.47 mg/L and 0.04 mg/L, respectively.

### Example 4: Analysis of Practical Environmental Water Samples

The method of the present invention was employed to analyze Cl⁻ and S²⁻ in practical environmental water samples. The Methylene Blue National Standard Method (GB/T16489-1996) was employed to analyze sulfur ions in practical environmental water samples, and the ion chromatography conductivity method was employed to analyze chlorine ions in practical environmental water samples. Five practical environmental water samples, which were identified as test sample A, B, C, D and E respectively were assayed. The steps of analysis were as follows:

### 1. Preparation of a Propelling Solution C

The propelling solution C was prepared according to step 3 of Example 2.

### 2. Preparation of a Color Developer Solution R

The color developer solution R was prepared according to step 4 of Example 2.

### 3. Processing of Test Samples

Nitric acid or sodium hydroxide was added into test sample A, B, C, D and E that had been filtered by medium-speed filter paper, to adjust the pH value of each test sample to 9-11. After the pH value of each test sample was well adjusted, each test sample was filtered using an aqueous microporous membrane with a pore diameter of 0.45 μm, and then decolorization was conducted using a macroporous absorption resin column.

### 4. Preparation of a Blank Sample

The blank sample was prepared according to step 2 of Example 1.

### 5. Mapping of Spectrogram of Test Samples

Steps (a) and (b) according to Step 5 of Example 2 were performed using each of test sample A, B, C, D and E that had been processed in step 3 instead of the standard sample S₂ in Example 2 to obtain spectrogram of S²⁻ and Cl⁻ of each of test sample A, B, C, D and E.

### 6. Assaying Results

The peak heights of S²⁻ and Cl⁻ in the spectrogram of each test sample mapped in step 5 were substituted into the regression equations of the standard working curves of sulfur ions and chlorine ions obtained in Example 3 to calculate the concentrations of S²⁻ and Cl⁻ in each test sample. The assaying results and the recovery rate of standard addition were shown in Tables 1 and 2.

TABLE 1

| | S²⁻ Assay | | | | |
|---|---|---|---|---|---|
| | | Measured Value/ | | | |
| | | (mg · L⁻¹) | | Recovery rate/% | |
| | Standard | | Methylene | | Methylene |
| | Addition/ | Present | Blue | Present | Blue |
| No. | (mg · L⁻¹) | Method | Method | Method | Method |
| A | 0 | 0 | 0 | | |
| | 5 | 4.51 ± 0.1 | 4.16 | 90.2 | 83.2 |
| | 10 | 9.44 ± 0.2 | 9.88 | 94.4 | 98.8 |
| B | 0 | 0.530 ± 0.05 | 0.500 | | |
| | 5 | 5.76 ± 0.07 | 5.25 | 105 | 95.0 |
| | 10 | 10.8 ± 0.6 | 10.3 | 103 | 97.7 |
| C | 0 | 2.13 ± 0.2 | 2.02 | | |
| | 5 | 6.77 ± 0.3 | 6.89 | 92.8 | 97.4 |
| | 10 | 11.6 ± 0.4 | 11.3 | 94.3 | 93.2 |
| D | 0 | 0.220 ± 0.1 | 0.250 | | |
| | 5 | 4.86 ± 0.3 | 5.13 | 92.8 | 97.6 |
| | 10 | 9.28 ± 0.1 | 9.91 | 90.6 | 96.6 |
| E | 0 | 0.820 ± 0.1 | 0.890 | | |
| | 5 | 5.33 ± 0.9 | 5.01 | 90.2 | 82.3 |
| | 10 | 9.83 ± 0.7 | 10.6 | 90.1 | 96.8 |

TABLE 2

| | Cl⁻ Assay | | | | |
|---|---|---|---|---|---|
| | | Measured Value/ | | | |
| | | (mg · L⁻¹) | | Recovery rate/% | |
| | Standard | | Ion Chroma- | | Ion Chroma- |
| | Addition/ | Present | tography | Present | tography |
| No. | (mg · L⁻¹) | Method | Method | Method | Method |
| A | 0 | 15.6 ± 0.86 | 15.9 | | |
| | 50 | 67.8 ± 1.3 | 65.7 | 104 | 99.6 |
| | 100 | 121 ± 0.72 | 114 | 106 | 98.1 |
| B | 0 | 46.8 ± 0.76 | 47.5 | | |
| | 50 | 103 ± 1.1 | 95.4 | 112 | 95.7 |
| | 100 | 152 ± 1.2 | 146 | 105 | 98.9 |
| C | 0 | 4.04 ± 0.57 | 4.17 | | |
| | 50 | 49.5 ± 0.86 | 47.5 | 90.8 | 96.7 |
| | 100 | 97.3 ± 0.95 | 98.3 | 93.3 | 94.3 |
| D | 0 | 8.55 ± 0.41 | 8.60 | | |
| | 50 | 53.1 ± 0.72 | 48.8 | 89.0 | 80.5 |
| | 100 | 109 ± 1.3 | 101 | 100.9 | 92.0 |
| E | 0 | 18.8 ± 0.49 | 19.4 | | |
| | 50 | 62.9 ± 1.0 | 60.3 | 88.2 | 81.7 |
| | 100 | 110 ± 1.5 | 110 | 91.5 | 90.6 |

What is claimed is:

1. A low pressure anion exchange chromatography—turbidimetric method for simultaneous online analysis of trace S²⁻ and Cl⁻ in water samples using an apparatus comprising a low pressure pump (1), a sample valve (2), a sample loop (3), a low pressure anion chromatographic column (4), a reactor (5), an optical flow cell (6), an optical detector (7), a computer system (8), a mixer (9), a sample flow path, a propelling solution flow path, and a color developer solution flow path, the method comprising:
(a) setting the apparatus in sample injection state, in which a blank sample (S₀) is driven by the low pressure pump (1) to enter the sample loop (3) through the sample flow path and the sample valve (2); and then setting the apparatus in analytical state, in which a color developer solution (R) is driven by the low pressure pump (1) to enter the mixer (9) through the color developer solution flow path, a propelling solution (C) is driven by the low pressure pump (1) to enter the sample loop (3) through the propelling solution flow path and the sample valve (2), the propelling solution brings the blank sample (S₀) in the sample loop (3) to enter the mixer (9) through the low pressure anion chromatographic column (4), they are mixed with the color developer solution in the mixer (9), then the mixture enters the optical flow cell (6) through the reactor (5), and signals produced via the optical detector (7) are transferred to the computer system (8) for processing to obtain a baseline;
(b) setting the apparatus in sample injection state, in which a test sample (S₁) is driven by the low pressure pump (1) to enter the sample loop (3) through the sample flow path and the sample valve (2); and then setting the apparatus in analytical state, in which the color developer solution (R) is driven by the low pressure pump (1) to enter the mixer (9) through the color developer solution flow path, the propelling solution (C) is driven by the low pressure pump (1) to enter the sample loop (3) through the propelling solution flow path and the sample valve (2), the propelling solution brings the test sample (S₁) in the sample loop (3) to enter the low pressure anion chromatographic column (4), S²⁻ and Cl⁻ in the test sample (S₁), after being separated in the low pressure anion chromatographic column (4), is brought by the propelling solution to enter the mixer (9) successively, where they are mixed with the color developer solution (R) respectively to form a first mixed solution and a second mixed solution, which enter the reactor (5) successively and form a first reaction solution and a second reaction solution upon color development reactions, the first reaction solution and the second reaction solution enter the optical flow cell (6) successively, and signals produced via the optical detector (7) are transferred to the computer system (8) for processing to obtain spectrogram of S²⁻ and Cl⁻ in the test sample (S₁);
(c) repeating steps (a) and (b) except for replacing the test sample (S₁) with a series of standard samples (S₂) in which the concentrations of S²⁻ and Cl⁻ are known, to obtain spectrogram of S²⁻ and Cl⁻ in the standard samples, and mapping standard working curves with the concentrations of S²⁻ and Cl⁻ in the standard samples being abscissa and the peak heights of S²⁻ and Cl⁻ in the spectrogram of S²⁻ and Cl⁻ in the standard samples being ordinate; and
(d) calculating the concentrations of S²⁻ and Cl⁻ in the test sample (S₁) by substituting the peak heights of S²⁻ and Cl⁻ in the spectrogram of S²⁻ and Cl⁻ in the test sample (S₁) into the regression equations of the standard working curves obtained in step (c), respectively;
wherein the test sample (S₁) and the standard samples (S₂) comprise NaOH in a concentration of from 10⁻⁵ mmol/L to 10⁻³ mmoL/L; the blank sample is an aqueous solution of NaOH in a concentration of from 10⁻⁵ mmol/L to 10⁻³ mmol/L; the propelling solution (C) is a mixed solution of nitric acid, sodium nitrate and deionized water; and the color developer solution (R) is a mixed solution of silver nitrate, polyvinylpyrrolidone K-30, gelatin, nitric acid and deionized water.

2. The method according to claim 1, wherein the propelling solution (C) comprises nitric acid in a concentration of 1-10 mmol/L, and sodium nitrate in a concentration of 1.0-10.0 g/L.

3. The method according to claim 1, wherein the color developer solution (R) comprises nitric acid in a concentration of 0.10-0.50 mol/L, silver nitrate in a concentration of 0.10-0.50 g/L, polyvinylpyrrolidone K-30 in a concentration of 0.10-0.50 g/L, and gelatin in a concentration of 0.50-1.0 g/L.

4. The method according to claim 2, wherein the color developer solution (R) comprises nitric acid in a concentration of 0.10-0.50 mol/L, silver nitrate in a concentration of 0.10-0.50 g/L, polyvinylpyrrolidone K-30 in a concentration of 0.10-0.50 g/L, and gelatin in a concentration of 0.50-1.0 g/L.

5. The method according to claim 1, wherein the detection wavelength of the optical detector is 420 nm.

6. The method according to claim 2, wherein the detection wavelength of the optical detector is 420 nm.

7. The method for simultaneous online analysis of trace S²⁻ and Cl⁻ in water samples according to claim 3, wherein the detection wavelength of the optical detector is 420 nm.

8. The method according to claim 4, wherein the detection wavelength of the optical detector is 420 nm.

9. The method according to claim 1, wherein the test sample (S₁) is filtrated by a microporous membrane and subjected to decolorization by macroporous adsorption resin before entering the low pressure pump (1).

10. The method according to claim 2, wherein the test sample (S₁) is filtrated by a microporous membrane and subjected to decolorization by macroporous adsorption resin before entering the low pressure pump (1).

11. The method according to claim 3, wherein the test sample (S₁) is filtrated by a microporous membrane and subjected to decolorization by macroporous adsorption resin before entering the low pressure pump (1).

12. The method according to claim 4, wherein the test sample (S₁) is filtrated by a microporous membrane and subjected to decolorization by macroporous adsorption resin before entering the low pressure pump (1).

13. The method according to claim 5, wherein the test sample (S₁) is filtrated by a microporous membrane and subjected to decolorization by macroporous adsorption resin before entering the low pressure pump (1).

14. The method according to claim 6, wherein the test sample (S₁) is filtrated by a microporous membrane and subjected to decolorization by macroporous adsorption resin before entering the low pressure pump (1).

15. The method according to claim 7, wherein the test sample (S₁) is filtrated by a microporous membrane and subjected to decolorization by macroporous adsorption resin before entering the low pressure pump (1).

16. The method according to claim 8, wherein the test sample (S₁) is filtrated by a microporous membrane and subjected to decolorization by macroporous adsorption resin before entering the low pressure pump (1).

17. The method according to claim 1, wherein the column filler of the low pressure anion chromatographic column is strongly basic quaternary ammonium anion exchange resin.

18. The method according to claim 17, wherein the particle size of the column filler is 30-35 μm.

19. The method according to claim 18, wherein the exchange capacity of the column filler is 3-4 mmol/g.
